(19) United States
(12) Patent Application Publication
(10) Pub. No.: US 2025/0222257 A1
(43) Pub. Date: Jul. 10, 2025

(54) DEVICE FOR AURICULAR PUNCTUAL STIMULATION

(71) Applicant: AURIMOD GMBH, Wien (AT)

(72) Inventors: Eugenijus KANIUSAS, Wien (AT); Stefan KAMPUSCH, Wien (AT); Florian THÜRK, Wien (AT)

(73) Assignee: AURIMOD GMBH, Wien (AT)

(21) Appl. No.: 18/851,555

(22) PCT Filed: Feb. 22, 2023
(86) PCT No.: PCT/IB2023/051610

§ 371 Date: Sep. 26, 2024

(30) Foreign Application Priority Data
Mar. 29, 2022 (EP) 22020137.0

(51) Int. Cl. A61N 1/36; A61N 1/04

(52) U.S. Cl. CPC A61N 1/36034; A61N 1/0456; A61N 1/36025; A61N 1/36031

(57) ABSTRACT

A device for auricular punctual stimulation of a patient, including an electrical current generator for generating electrical stimulation pulses and electrical lines for connecting to one electrode each to be positioned on the ear, at least two of which electrodes can be acted on by the stimulation pulses, and further including measurement means for determining at least one physiological measured value of the patient. The measurement means are designed to determine the at least one physiological measured value by means of impedance plethysmography via the electrodes.

### CROSS-REFERENCE TO RELATED APPLICATIONS

The present application is a national phase application of PCT Application No. PCT/IB2023/051610, filed Feb. 22, 2023, entitled “DEVICE FOR AURICULAR PUNCTUAL STIMULATION”, which claims the benefit of European Patent Application No. 22020137.0, filed Mar. 29, 2022, each of which is incorporated by reference in its entirety.

### BACKGROUND OF THE INVENTION

### 1. Field of the Invention

The invention relates to a device for auricular punctual stimulation of a patient, comprising an electrical current generator for generating electrical stimulation pulses and electrical lines emanating from the electrical current generator for connecting to one electrode each to be positioned on the ear, at least two of which can be acted on by the electrical stimulation pulses, and further comprising measurement means for determining at least one physiological measured value of the patient.

### 2. Description of the Related Art

Available stimulation apparatus for electrical stimulation, e.g. of the auricular vagus nerve, are used in the therapy of a wide variety of diseases, such as chronic and acute pain, epilepsy and depression. In auricular punctual stimulation apparatus, as have become known, for example, from WO 2011/030210 A1, electrical stimulation pulses are introduced via needle electrodes, which are pierced into the skin at predetermined points of the auricle and remain there for the treatment period of, for example, several days.

Stimulation makes it possible, for example, to advantageously influence pain processing and pain perception. The sympathovagal balance in the autonomic nervous system is also positively influenced by the stimulation. As a result, the current physiological state of the patient changes dynamically, measured as changes in brain activity, heart rate, respiratory rate, blood pressure, local blood flow, and other parameters.

In current technical solutions, the stimulation parameters either are fixed or can be adjusted by the doctor and/or patient during application or readjustment of the device. The parameters therefore do not or only very poorly consider the current physiological state of the patient, which may change greatly over the therapeutic process. Lack of physiological feedback causes overstimulation or understimulation of the patient over the entire therapeutic period, whereby critical therapeutic goals are missed. For example, amplitude, frequency, switch-on and switch-off times cannot be optimally controlled. Overstimulation can cause additional pain, with the further disadvantage that the battery of the device is discharged more quickly. Understimulation disregards the individual distress of the patient and thus misses the critical therapeutic goals. For example, the nerve is not sufficiently stimulated to achieve the desired therapeutic effect.

Currently available concepts for adaptive stimulation apparatus additionally use implanted or internal and external sensors to realise a physiological feedback signal for the individualization of the therapy. Cardiovascular and cardiorespiratory parameters can be used for targeted control or even for feedback control of the instantaneous stimulation parameters in order to enable disease-and patient-specific therapy by means of feedback-based stimulation.

A major disadvantage of existing concepts of stimulation apparatus is the need for at least one internal or external sensor (e.g. PPG sensor, etc.) that provides a physiological sensor signal for individualized control of stimulation. External sensors, in particular, are not only a hindrance to the patient, but also represent a significant expenditure of time and cost factor for the training and at the beginning and implementation of therapy. The possibilities of internal sensors are often limited with regard to the possibility of capturing meaningful data, or they also need additional space and energy.

### SUMMARY OF THE INVENTION

The present invention therefore aims to improve a device for auricular punctual stimulation, e.g. of the vagus nerve, in such a way as to simplify the determination of a physiological measured value, in particular avoiding the disadvantages mentioned above.

To achieve this object, the invention essentially consists in a device of the type mentioned before in that the measurement means are configured to determine the at least one physiological measured value by means of impedance plethysmography via the electrodes. By determining the physiological measured value using the principle of impedance plethysmography, the electrodes already present for stimulation of the vagus nerve, for example, can be also used to measure the tissue impedance in the ear. Thus, a sensor implementation was found that does not require additional sensors that would have to be implanted or that would have to be attached to a measuring point on the body. This avoids impairment of the patient by additional sensors and thus additionally improves patient compliance. Furthermore, the expenditure of time and costs for the training as well as at the beginning and implementation of therapy are reduced. However, the invention is not limited to the fact that the stimulation and the impedance measurement take place via the same electrodes. On the contrary, electrodes other than for the stimulation can be used for the impedance measurement. Preferably, however, at least one electrode through which the stimulation takes place is also used for the impedance measurement.

Impedance plethysmography is a method by which the electrical AC resistance, i.e. the impedance, of a body part can be detected. Since blood is a good electrical conductor in comparison to other types of tissue, changes in the blood volume at the measuring point lead to measurable impedance changes. For impedance measurement, a high-frequency alternating current is impressed into the ear via two electrodes, which is subthreshold and therefore does not irritate the auricular nerves. The impedance is usually measured by tapping the voltage between two electrodes, which changes depending on the blood flow. Consequently, in this way the blood flow can be detected and analyzed.

A preferred embodiment of the device according to the invention provides in this context that the electrical current generator is configured to generate an alternating current, which can be introduced into the ear via two of the electrical lines and the associated electrodes, and that the measurement means have a measuring circuit for measuring a tissue impedance tapped between two electrodes.

In this case, the measurement of impedance can be done using two, three or four electrodes or more. In the case of three electrodes, comprising two external electrodes and a central electrode arranged between them, the introduction of the alternating current advantageously takes place via the external electrodes, and the voltage tapping takes place via the central and one of the two external electrodes. In the case of four electrodes, comprising two external electrodes and two central electrodes arranged along the current path between them, the introduction of the alternating current advantageously is done via the external electrodes and the voltage tapping is done via the two central electrodes.

The impedance measurement can be done in such a way that an alternating current is introduced at a predetermined current intensity and the voltage drop is determined at the measuring electrodes. Alternatively, alternating current can be introduced at a predetermined voltage and the current intensity resulting via the measuring electrodes can be determined.

The local tissue impedance and its dynamic changes reflect local perfusion conditions in the ear and thus allow individual and time-varying information about the patient's cardiac and respiratory situation.

The at least one physiological measured value determined with the device according to the invention may be a primary measured value or a derived, secondary measured value. The primary measured value is the tissue impedance, which is proportional to the voltage tapped across the electrodes and can be detected by the measuring circuit. Derived measured values can be determined from the tissue impedance or from the time course of the tissue impedance. This is achieved, for example, by using signal processing methods comprising maximum and minimum value detection, frequency filters, Fourier analysis, and wavelet transformations. Examples of derived measured values comprise heart rate, heart rate variability, blood flow, vascular stiffness, and respiratory rate. In this case, the signal processing can be done in the device according to the invention or in an external device provided with the primary measured values.

In the first case a preferred development of the invention provides that the measurement means include a signal processing circuit which is connected to the measuring circuit and which is configured to determine the at least one physiological measured value, such as the heart rate, the heart rate variability, the blood flow, the vascular stiffness and/or the respiratory rate, from the time course of the tissue impedance. These are measured values that allow conclusions to be drawn about the state of the vegetative nervous system of the patient and the effect of the stimulation therapy. Heart rate variability, for example, is a surrogate parameter for the sympathovagal balance or condition of the autonomic nervous system of the patient.

The at least one physiological measured value and its temporal change can be used to observe and record the therapeutic success of the punctual stimulation, e.g. of the vagus nerve, for example over several weeks. The recording of the at least one physiological measured value and of its temporal change can serve documentation purposes only. However, it is preferably provided that the at least one physiological measured value or its temporal change is used to control the stimulation therapy, e.g. in the sense of increased stimulation frequency and/or amplitude when the result is poor or reduction of these parameters when the result is good. The measured values can be used, for example—in the simplest case—as adaptive threshold values for the individual increase or reduction of the stimulation strength or the stimulation frequency. For example, severe pain increases the current heart rate, which could then lead to increased nerve stimulation and thus to increased pain relief due to the physiological feedback.

For this purpose, a preferred development of the device according to the invention provides that a control circuit is provided, which interacts with the electrical current generator to change at least one electrical stimulation parameter, such as the pulse frequency, the length of a burst of stimulation pulses, the current amplitude and/or the duty cycle of the electrical stimulation pulses. Particularly preferably the measurement means interact with the control circuit to adjust the at least one electrical stimulation parameter as a function of the at least one physiological measured value.

The electrical stimulation parameters can be adjusted, for example, in such a way that when the heart rate is increased or the heart rate variability is reduced, the stimulation frequency or burst length is increased, or the duty cycle is increased, i.e. the switch-on duration is increased and/or the switch-off duration is reduced. Stimulation can also be triggered by certain events, e.g. stimulation can take place in the rhythm of the heart rate or only during exhalation.

The electrical stimulation pulse sequence used for nerve stimulation and the alternating current used for tissue impedance measurement do not necessarily have to differ from each other. The stimulation current can thus also be used to measure the tissue impedance if it is configured as an alternating current.

Preferably, however, the electrical stimulation pulse sequence used for nerve stimulation and the alternating current used for measuring the tissue impedance may differ from one another with regard to the different effect to be achieved, at least with regard to their current amplitude and their frequency. For impedance measurement, a significantly higher frequency and a lower current amplitude is advantageous than for nerve stimulation. The electrical current generator is therefore suitable for generating different current amplitudes and frequencies.

Preferably, it is provided here that the electrical current generator is configured to generate the alternating current with an alternating current frequency of 5-100 kHz. In contrast, the electrical current generator is preferably configured to generate the electrical current pulses with a pulse frequency of <1 kHz.

With respect to the current amplitude, it is preferably provided that the electrical current generator is configured to generate the alternating current with a current amplitude of <2 mA. Depending on the frequency and electrode shape, such a low current is below the stimulation threshold, e.g. of the vagus nerve. For the purposes of the stimulation, the electrical current generator is preferably configured to generate the electrical current pulses with a current amplitude of >5 mA. In this case, the electrical stimulation pulses may preferably have an alternating polarity.

However, since the stimulation threshold depends on numerous factors, absolute limit values of current amplitude are not always applicable. According to an alternative embodiment it is therefore provided that the electrical current generator is configured to generate the electrical stimulation pulses with a current amplitude that is at least 2 times, preferably at least 3 times, the current amplitude of the alternating current.

The electrical stimulation pulses and the alternating current used for impedance measurement can also differ with regard to the pulse or waveform. The alternating current for measurement can preferably have sinusoidal shape. The electrical stimulation pulses, on the other hand, can have a rectangular shape in the sense of a rectangular wave.

The impedance measurement and the nerve stimulation can preferably be carried out alternately in order to avoid a mutual interference of the two processes. Here a preferred embodiment of the device according to the invention provides that the electrical current generator is configured to generate a plurality of sequences of electrical stimulation pulses with pauses between the sequences and to generate the alternating current in at least one of the pauses.

Alternatively, an additional sub-threshold alternating current can be applied during nerve stimulation, so that the electrical stimulation pulses are superimposed with the alternating current used for impedance measurement. Thereby, the resulting additional overvoltage is measured in order to calculate the tissue impedance from the ratio of the two.

As already mentioned, the at least one physiological measured value, i.e. the tissue impedance or a value derived therefrom, such as the heart rate, the heart rate variability, the vascular filling, the respiratory rate and the like, can be evaluated in the device itself or in an external device and, if necessary, used as a controlled variable for adjusting the nerve stimulation parameters. For the external evaluation, the device according to the invention is preferably configured such that the device includes a communication interface to which the at least one physiological measured value or its temporal change is supplied and which is configured to transmit the at least one physiological measured value or its temporal change to an external receiving device.

In order to enable a bidirectional communication the communication interface is preferably configured to receive control commands that can be supplied to the control circuit in order to adjust the electrical stimulation parameters as a function of the control commands.

### BRIEF DESCRIPTION OF THE DRAWINGS

The invention will be explained in more detail below with reference to exemplary embodiments schematically illustrated in the drawings.

FIG. 1 shows a schematic illustration of the device according to the invention,

FIG. 2 shows the arrangement of needle electrodes on a human ear with an illustration of voltage and current for vagus nerve stimulation,

FIG. 3 shows the arrangement of needle electrodes on a human ear with a illustration of voltage and current for tissue impedance measurement,

FIG. 4 shows a sequence of electrical stimulation pulses and the alternating current, and

FIG. 5 shows the signal obtained from the tissue impedance measurement.

### DETAILED DESCRIPTION

FIG. 1 shows an exemplary embodiment of the device according to the invention comprising a current generator 1, which is fed by a battery (not shown), to which electrical lines 2, 3 and 4 are connected, at each of whose ends a needle electrode 5, 6 and 7 to be positioned on the ear is arranged. The current generator 1 is configured to generate electrical stimulation pulses, which are introduced into the ear via the lines 2, 3, 4 and the associated electrodes 5, 6, 7. Furthermore, the current generator 1 is configured to generate an alternating current, which can be introduced into the ear, for example, via the electrical lines 2 and 4 and the corresponding electrodes 5 and 7. A measuring circuit 8 is provided for measuring the tissue impedance tapped, for example, via the electrodes 6 and 7.

To a measurement circuit 8 a signal processing circuit 9 is connected which is supplied with the measured values of measuring circuit 8 and which is configured to determine at least one physiological measured value, such as the heart rate, the heart rate variability, the blood flow, the vascular stiffness and/or the respiratory rate, from the time course of the tissue impedance. Furthermore, a control circuit 10 is provided, which interacts with the current generator 1 to change at least one electrical stimulation parameter, such as the pulse frequency and/or the current amplitude of the electrical stimulation pulses. In this case, the at least one electrical stimulation parameter can be changed as a function of the tissue impedance or of the physiological measured value determined by the signal processing circuit 9 or of its time course, for which purpose the measured value is supplied to the control circuit 10 by the signal processing circuit 9.

The current generator 1, the measuring circuit 8, the signal processing circuit 9, and the control circuit 10 are arranged in a housing 11 that can be attached near the ear, e.g., behind the ear. Alternatively, an attachment to another part of the body, such as the chest, is also conceivable. The current generator 1, the measuring circuit 8, the signal processing circuit 9 and the control circuit 10 can be configured as separate components or be implemented in a common electronic circuit.

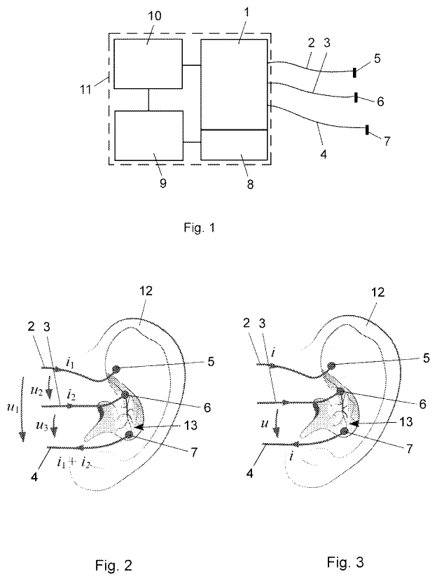

FIG. 2 shows the human ear 12 with blood vessels and the afferent vagus nerve branches 13. In the region of the vagus nerve branches, the needle electrodes 5, 6 and 7 are pierced into the tissue, a sequence of stimulation pulses with the current i₁ being introduced via the line 2 and a sequence of stimulation pulses with the current i₂ being introduced via the line 3, and the current return flow i₁+i₂ taking place via the line 4. A voltage u₁ results between the lines 2 and 4, a voltage u₂ results between the lines 2 and 3, and a voltage u₃ results between the lines 3 and 4.

FIG. 3 shows the measurement of the tissue impedance in the ear by means of an alternating current. Thereby, an alternating current circuit is generated via the lines 2 and 4, and the voltage u is tapped via the lines 3 and 4 and measured in the measuring circuit 8.

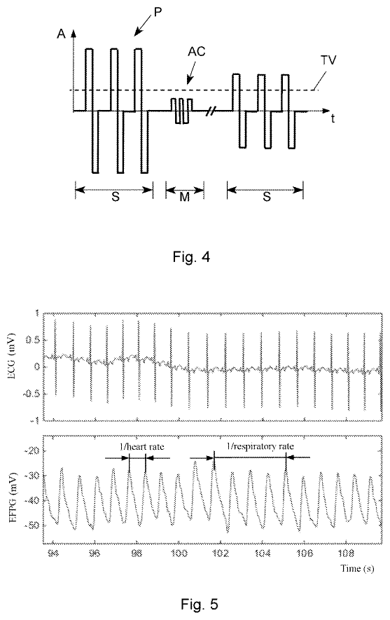

FIG. 4 shows the course of a current with the current intensity or voltage A over time t. In a first stimulation phase S, the electrical current generator 1 generates a sequence of electrical stimulation pulses P, which have the shape of rectangular waves and are above the threshold value TV, the exceeding of which triggers a stimulation stimulus. In a subsequent measurement phase M, a subthreshold alternating current AC is generated, which is used for tissue impedance measurement. This is followed by another phase S with a sequence of electrical stimulation pulses P.

FIG. 5 shows an exemplary recording of a bahavior of the local tissue impedance compared to a synchronously recorded electrocardiogram (ECG). The impedance cardiogram was derived via three stimulation electrodes attached to the ear, as illustrated in FIG. 3. The cardiac activity as well as the respiratory activity are clearly recognizable and can thus be used for sensorless control of the auricular stimulation.

1-14. (canceled)

15. A device for auricular punctual stimulation of a patient, comprising:
an electrical current generator configured to generate electrical stimulation pulses;
electrical lines for connecting to one electrode each to be positioned on the ear, at least two of which electrodes can be acted on by the electrical stimulation pulses; and
measurement means for determining at least one physiological measured value of the patient, the measurement means being configured to determine the at least one physiological measured value by means of impedance plethysmography via the electrodes;
wherein the electrical current generator is configured to generate an alternating current, which can be introduced into the ear via two of the electrical lines and the associated electrodes;
wherein the measurement means have a measuring circuit for measuring a tissue impedance tapped between two electrodes; and
wherein the current generator is configured to generate a plurality of sequences of electrical stimulation pulses with pauses between the sequences and to generate the alternating current in at least one of the pauses.

16. The device according to claim 15, wherein the measurement means include a signal processing circuit which is connected to the measuring circuit and which is configured to determine the at least one physiological measured value from the time course of the tissue impedance.

17. The device according to claim 16, wherein the at least one physiological measured value comprises at least one of a heart rate, a heart rate variability, a blood flow, a vascular stiffness and a respiratory rate.

18. The device according to claim 15, wherein the electrical current generator is configured to generate the alternating current with an alternating current frequency of 5-100 kHz.

19. The device according to claim 15, wherein the electrical current generator is configured to generate the electrical stimulation pulses with a pulse frequency of <1 kHz.

20. The device according to claim 15, wherein the electrical current generator is configured to generate the alternating current with a current amplitude of <2 mA.

21. The device according to claim 15, wherein the electrical current generator is configured to generate the electrical stimulation pulses with a current amplitude of >5 mA.

22. The device according to claim 15, wherein the electrical current generator is configured to generate the electrical stimulation pulses with a current amplitude at least 2 times a current amplitude of the alternating current.

23. The device according to claim 22, wherein the current amplitude of the electrical stimulation pulses are at least 3 times the current amplitude of the alternating current.

24. The device according to claim 15, wherein a control circuit is provided, which interacts with the electrical current generator to change at least one electrical stimulation parameter.

25. The device according to claim 24, wherein the at least one electrical stimulation parameter comprises a pulse frequency, a length of a burst of stimulation pulses, a current amplitude and a duty cycle of the electrical stimulation pulses.

26. The device according to claim 24, wherein the measurement means interact with the control circuit to adjust the at least one electrical stimulation parameter as a function of the at least one physiological measured value.

27. The device according to claim 15, wherein the electrical current generator is configured to vary the frequency of the alternating current to allow a dispersion depending characterisation of the tissue impedance.

28. The device according to claim 15, wherein the device includes a communication interface to which the at least one physiological measured value or its temporal change is supplied and which is configured to transmit the at least one physiological measured value or its temporal change to an external receiving device.

29. The device according to claim 15, wherein the communication interface is configured to receive control commands that can be supplied to the control circuit in order to adjust the electrical stimulation parameters as a function of the control commands.
